(19) United States
(12) Patent Application Publication
(10) Pub. No.: US 2008/0051770 A1
(43) Pub. Date: Feb. 28, 2008

(54) Multiple Target Laser Probe

(75) Inventors: Gregg D. Scheller, Glencoe, MO (US); James C. Easley, Cottleville, MO (US)

Correspondence Address: THOMPSON COBURN, LLP, ONE US BANK PLAZA, SUITE 3500, ST LOUIS, MO, 63101, US

(73) Assignee: SYNERGETICS, INC., O'Fallon, MO (US)

(21) Appl. No.: 11/843,433

(22) Filed: Aug. 22, 2007

Related U.S. Application Data
(60) Provisional application No. 60/823,181, filed on Aug. 22, 2006.

(51) Int. Cl. A61B 18/20

(52) U.S. Cl. 606/4

(57) ABSTRACT

A multiple target ophthalmic surgery instrument is comprised of a single primary optic fiber that transmits laser light to the instrument, and a plurality of additional optic fibers that receive the laser light from the primary optic fiber and project the laser light as a plurality of beams from the plurality of additional optic fibers. In this manner, the instrument splits the single laser light beam received from a single laser light source into the multiple of laser beams and targets the multiple laser beams at multiple spots of a surgical site in the eye.

This patent application claims the priority benefit from the provisional patent application Ser. No. 60/823,181, filed on Aug. 22, 2006, which is incorporated by reference herein.

### BACKGROUND OF THE INVENTION

(1) Field of the Invention

The present invention pertains to a microsurgical laser probe used primarily in ophthalmic surgery. The laser light delivered to the probe is split and projected as multiple laser light beams from the probe. The split multiple beams are directed to multiple spots at a surgical site to provide laser light treatment at each spot.

(2) Description of the Related Art

Various different types of lasers are used in medicine to treat a number of pathologies. The interaction of laser light with body tissue is effected by the wavelength of the laser light, as well as the power density of the light. Additionally, the presence of drugs or dies on the body tissue along with prior light exposure will influence the effectiveness of the laser light treatment.

Light radiation treatment patterns on body tissue are chosen based on the desired result. In some instances, for example activation of a drug in a photodynamic therapy, the desired radiation pattern of laser light on body tissue is broad and diffuse. This allows for laser light treatment in a large area of the body tissue without concern for variations in the power density of the light.

Other instances may require relatively small intense treatment areas of laser light on the body tissue, for example in pan retinal photocoagulation in retinal surgery. In these applications, a relatively small beam of laser light with relatively high-power density is directed to a treatment area of the body tissue to ablate tissue or to cause a small burn.

Specifically in retinal surgery, a small beam of laser light is used to produce small burn spots on the retina that help to adhere the retina in place after a retinal detachment, or after a hole has occurred. Coagulation of the retinal tissue at the treatment spots forms an initial bond, and scar tissue maintains that bond after healing.

It is common to apply well over 500 laser light treatment spots on the retina during a retinal surgery procedure. A solid state laser operating at 532 nm is the most common laser light source currently used. An endophotocoagulation probe with a 200 or 300 micron optical fiber is the normal delivery instrument for the laser light beam. The probe tip is inserted through a sclerotomy into the eye and is directed at the retinal surface where a burn is desired. A treatment pulse of approximately 0.25 watts for approximately 0.25 seconds is delivered to the retinal tissue to cause a burn spot that coagulates the tissue. The laser light probe is manually aimed by the surgeon and fired repeatedly producing a multiple of burn spots on the retina until the surgeon feels that the retina has been properly treated, i.e., a coagulation bond has been produced in the retina tissue.

The time needed to deliver the large number of laser light pulses that produce the multiple treatment spots on the retina is a disadvantage. What is needed to overcome this disadvantage is an instrument that reduces the time required to deliver laser light treatment without sacrificing the quality of that treatment.

### SUMMARY OF THE INVENTION

The multiple target laser probe apparatus of the invention overcomes the disadvantages of the prior art by providing an ophthalmic surgery instrument that is capable of splitting a single laser beam received from a single laser light source into a multiple of laser beams, and to target the multiple laser beams at multiple spots of a surgical site in the eye. In this manner, the apparatus of the invention enables ophthalmic surgery procedures to be performed in a more time-efficient manner.

The apparatus is provided in several different embodiments that each multiply a laser light beam provided from a single laser light source and direct the multiple laser light beams in a predetermined pattern. A preferred embodiment of the apparatus includes a manually manipulatable instrument that includes a handle with a tubular tip projecting from the handle, a single primary optic fiber that extends into the handle, and a plurality of secondary optic fibers that extend through the instrument tip.

The primary optic fiber has a light source connector at an opposite end of the fiber from the instrument. Connecting the light source connector to a separate light source enables laser light to be transmitted through the length of the primary optic fiber to the instrument.

In the preferred embodiment, the end of the primary optic fiber received by the instrument is connected inside the instrument to the ends of the plurality of secondary optic fibers that extend through the instrument tip. The connection allows laser light transmitted through the primary optic fiber to be transferred to each of the plurality of secondary optic fibers and transmitted through the lengths of the secondary optic fibers.

The light transferred to the secondary optic fibers is transmitted through the lengths of the secondary optic fibers to distal end surfaces of the secondary optic fibers positioned adjacent the distal end of the instrument tip. A spacer inside the instrument tip maintains the distal ends of the plurality of additional optic fibers in their relative positions. The distal end surfaces of the plurality of secondary optic fibers project beams of laser light in a spaced relationship and in a plurality of projection directions from the instrument tip.

In an alternate embodiment, the plurality of secondary fibers extend through the length of the handle and include at their ends opposite the tip a connector, which is used to connect the plurality of fibers of the instrument to a laser light source.

In a further embodiment of the instrument, the plurality of secondary optic fibers are eliminated and the primary optic fiber extends through the instrument handle and the instrument tip. A small portion of the primary optic fiber distal end projects outwardly from the distal end of the instrument tip. A pyramid shape with four polished surfaces is formed on the distal end portion of the primary optic fiber. Laser light that travels through the primary optic fiber strikes each of the four flat polished surfaces and is reflected to an opposite surface of the four flat polished surfaces at the optic fiber distal end. The reflected laser light striking the opposite flat surfaces of the optic fiber distal end is refracted through the surfaces and exits the distal end of the optic fiber as four laser light beams. Due to the relative angles of the four flat surfaces at the optic fiber distal end, the four individual laser light beams diverge slightly from each other as they are directed away from the distal end of the instrument tip.

A still further embodiment of the instrument of the invention employs the primary optic fiber that extends through the instrument handle and the instrument tip to a distal end of the optic fiber that is positioned adjacent the distal end of the instrument tip, but inside the instrument tip. A glass disk is positioned inside the instrument tip at the tip distal end. The glass disk has an exterior end surface that is formed with a plurality of micro lens surfaces. The lens surfaces are convex surfaces that gather the laser light striking the lens surfaces inside the glass disk and focus the laser light into separate laser light beams that are projected from the convex exterior surface of each of the lenses.

Each of the embodiments of the ophthalmic surgery instrument described above is capable of splitting a single laser light beam received from a single laser light source into a multiple of laser light beams that can be directed by the instrument to a multiple of spots at a surgical site in the eye. In this manner, the embodiments of the apparatus of the invention enable ophthalmic surgery procedures to be performed in a more time efficient manner.

### BRIEF DESCRIPTION OF THE DRAWINGS

Features of the invention are set forth in the following description of the preferred embodiments of the invention and in the drawing figures of the application.

FIG. 1 is a side elevational view, partially in section, of a first embodiment of the apparatus of the invention.

FIG. 2 is an enlarged partial view, in section, of a portion of the apparatus shown circled in FIG. 1.

FIG. 3a is an enlarged partial view, in section, of a portion of the apparatus shown circled in FIG. 1.

FIG. 3b is a right side end view of FIG. 3a.

FIG. 4a is an enlarged view of a variant embodiment of the distal end of the apparatus tip.

FIG. 4b is a right side end view of FIG. 4a.

FIG. 5 is a side elevation view, partially in section, of a second embodiment of the apparatus.

FIG. 6 is an enlarged partial view, in section, of a portion of the apparatus shown circled in FIG. 5.

FIG. 7 is an enlarged perspective view of the distal end of the optic fiber of the apparatus shown in FIG. 5.

FIG. 8 is a side elevation view, partially in section, of a third embodiment of the apparatus.

FIG. 9 is an enlarged partial view, in section, of a portion of the apparatus shown circled in FIG. 8.

FIG. 10 is an enlarged perspective view of a distal end of the tip of the apparatus shown in FIG. 8.

### DESCRIPTION OF THE PREFERRED EMBODIMENTS

The present invention provides a microsurgical instrument that is capable of splitting a single laser beam received from a single laser light source into a multiple of laser beams, and to target the multiple laser beams at multiple spots of a surgical site in the eye. In this manner, the apparatus of the invention enables ophthalmic surgery procedures to be performed at multiple spots within the surgical site at the same time.

The apparatus is provided in several different embodiments that each multiply a laser light beam transmitted from a single laser light source to the apparatus into a multiple of laser light beams, and direct the multiple laser light beams in a predetermined projection pattern at an ophthalmic surgery site.

In general, the construction of each embodiment is somewhat similar to that of instruments disclosed in the Scheller U.S. Pat. No. 5,785,645 and the et al. Scheller U.S. Pat. No. 5,807,242, both of which are incorporated by reference.

Each embodiment of the apparatus is a manually manipulatable instrument comprising an elongate handle 12 dimensioned to be easily gripped and maneuvered by a single hand of the surgeon. In a preferred embodiment, the handle 12 has a center bore 14 that extends through the entire length of the handle.

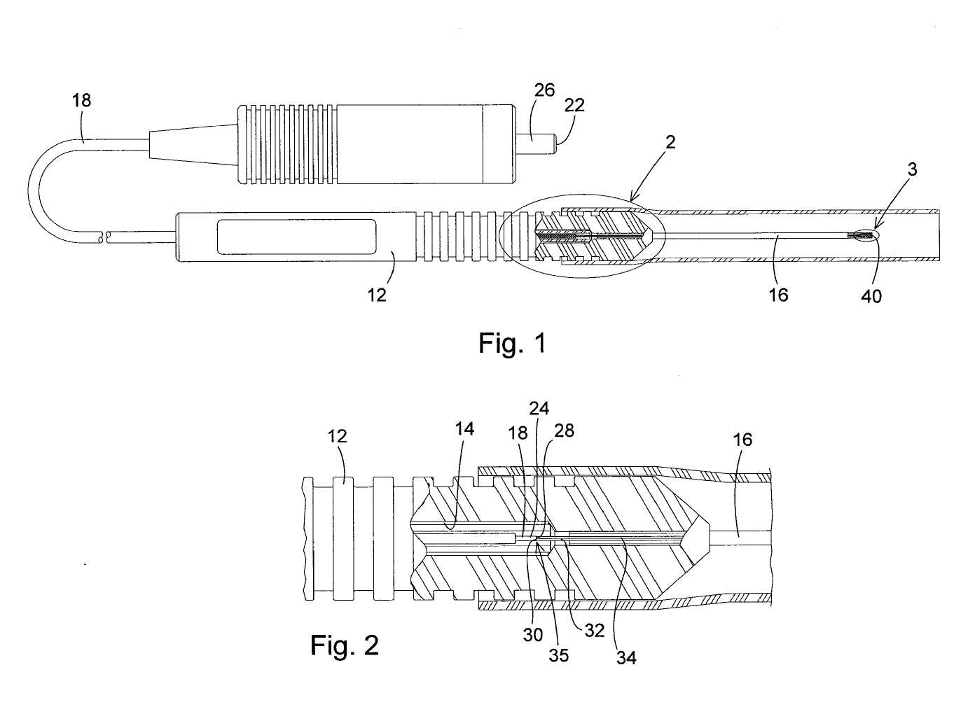

In FIG. 1, a rigid tubular tip 16 projects from a distal end of the handle 12. The handle bore 14 communicates with the interior of the tip. The tip 16 is preferably constructed of surgical stainless steel and has the dimensions of a syringe needle. These minimal dimensions are needed for ophthalmic surgery.

In an alternate embodiment the tip 16 is flexible, as is described in U.S. Pat. Nos. 6,572,608 and 6,984,230, both of which are incorporated by reference.

A length of optic fiber 18 having opposite proximal 22 and distal 24 ends is connected to the handle 12. A conventional laser light source connector 26 is provided on the optic fiber proximal end 22. The connector 26 is adapted to be removably connected to a socket of a commercially available laser light source. The optic fiber distal end 24 enters the handle 12 and extends through the handle bore 14. When the connector 26 is connected to the laser light source, laser light is transmitted through the length of the optic fiber to the end surface 28 of the optic fiber at the optic fiber distal end 24.

In a first embodiment of the invention, the optic fiber 18 is a first or primary optic fiber of a multiple of optic fibers employed by the apparatus. The primary optic fiber distal end 24 is positioned in the handle interior bore 14. In the handle bore, the distal end 24 of the primary optic fiber 18 is connected to the proximal ends 32 of a plurality or multiple of additional optic fibers 34. This joint between the primary optic fiber 18 and the additional optic fibers 34 can be positioned at other locations within the apparatus, and need not be located in the instrument. In the drawing figures, four additional optic fibers 34 are shown. However, the instrument could comprise a greater or lesser number of additional optic fibers 34. The connection between the primary optic fiber 18 and the additional optic fibers 34 allows laser light transmitted through the primary optic fiber 18 to pass from the distal end surface 28 of the primary optic fiber 18, through the proximal end surfaces 30 of the additional optic fibers 34, and be transmitted by the additional optic fibers 34. In this way, the apparatus of the invention splits the laser light transmitted by the primary optic fiber 18 as the laser light enters the plurality of additional optic fibers 34.

There are a number of ways in which the distal end surface 28 of the primary optic fiber 18 can be connected to the proximal end surfaces 30 of the additional optic fibers 34 to split the laser light transmitted by the primary optic fiber 18 into the plurality of additional optic fibers 34. Each of these different methods of joining the primary optic fiber distal end surface 28 to the proximal end surfaces of the additional optic fibers 30 is represented by the connection 35 schematically represented in FIG. 2. In an embodiment, the connection 35 of the primary optic fiber 18 to the additional optic fibers 34 can be provided by a fused glass joint. Alternatively, an adhesive could provide the connection 35. Still further, the connection 35 could be provided by an index of refraction matching gel. Use of such a gel to form the connection 35 aids in reducing light loss at the connection. The connection 35 provided by an index of refraction matching gel could be contained in a tubular glass capillary that would surround the gel and hold the gel in position at the connection 35. As a still further embodiment of the connection 35, the index of refraction matching gel could be contained in a tube made of a material such as aluminum or another similar heat conducting material. Employing this type of material in the connection 35 would allow the heat energy of any stray laser light escaping the connection between the primary optic fiber 18 and the additional optic fibers 34 to be absorbed by the heat conducting material of the tube and dissipated by the tube.

The additional optic fibers 34 all extend from the handle interior bore 14 and into the tubular tip 16. The additional optic fibers extend through the tubular tip 16 to distal ends 36 of the additional optic fibers 34. Laser light transmitted through the plurality of additional optic fibers 34 is projected from the distal end surfaces 38 of the additional optic fibers that are positioned adjacent the distal end 40 of the tip 16.

A spacer insert 42 is provided inside the tubular tip 16 adjacent the tip distal end 40. The spacer insert 42 is generally cylindrical and has a plurality of channels 43 that receive the distal ends 36 of the additional optic fibers 34 and hold the distal ends 36 apart from each other in a spaced relationship around the interior circumference of the tip distal end 40. In this manner, the spacer insert 42 determines the pattern of the multiple laser light beams projected from the distal end surfaces 38 of the plurality of additional optic fibers 34 at the tip distal end 40.

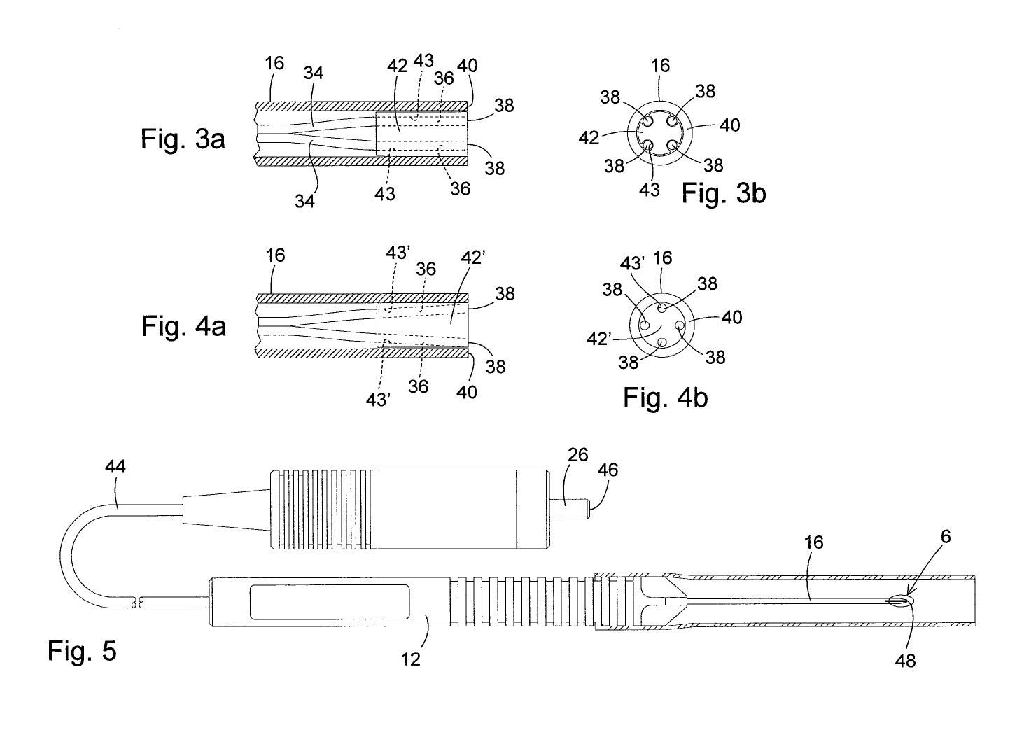

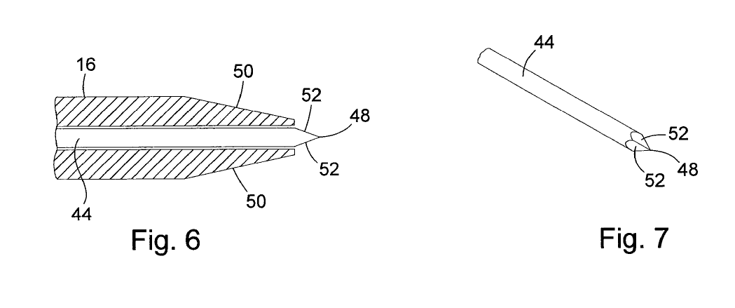

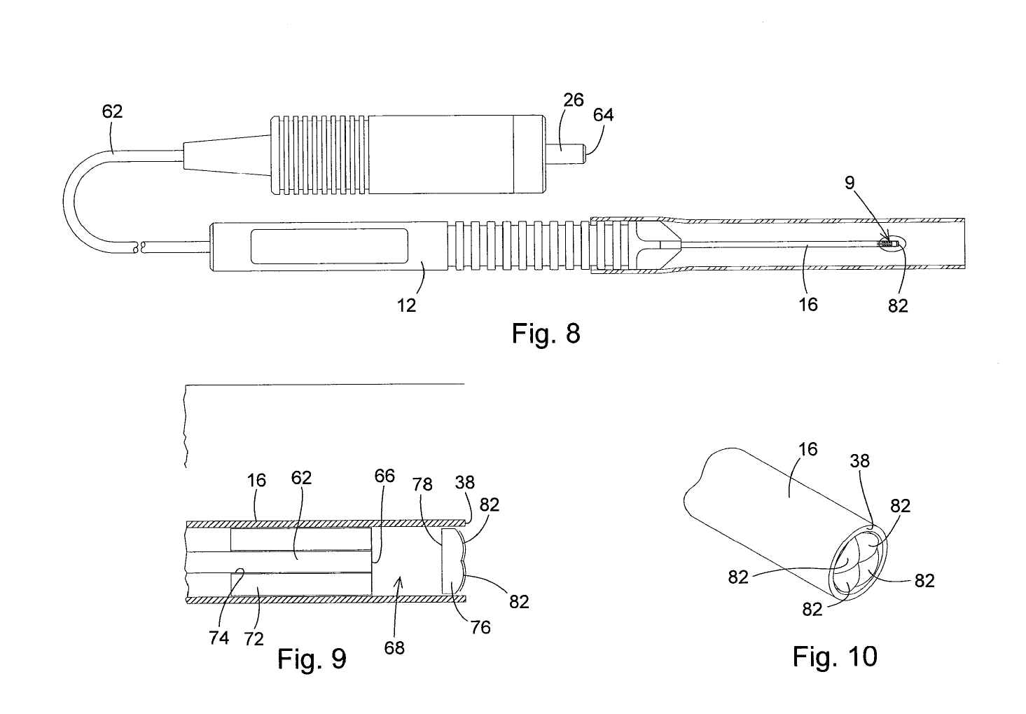

FIGS. 3a and 3b show the positioning of the distal ends 36 of the additional optic fibers 34 held by the spacer 42 in the instrument tip 16. The distal ends of the additional optic fibers 34 are held by the spacer 42 in a spaced relationship from each other around the interior of the instrument tip 16 adjacent the tip distal end 40. Thus, laser light projected from each of the distal end surfaces 38 of the plurality of additional optic fibers 34 will be directed in one direction from the tip distal end 40. With the spacing of the distal ends 36 of the additional optic fibers 34 held by the spacer 42, the laser light projected from the distal end surfaces 38 of the additional optic fibers 34 may diverge slightly from the plurality of fibers, but in the preferred embodiment the individual beams projected from the distal end surfaces 38 will not merge into a single spot.

FIGS. 4a and 4b show a variation of the spacer shown in FIGS. 3a and 3b. However, the channels 43′ of the spacer 42′ shown in FIGS. 4a and 4b do not extend parallel to each other through the spacer as do the channels 43 in the embodiment of FIGS. 3a and 3b, but diverge slightly from each other as they extend through the length of the spacer 42′ to the tip distal end 40. The distal ends 36 of the additional optic fibers 34 held by the spacer 42′ shown in FIGS. 4a and 4b will project laser light beams in a diverging pattern from the tip distal end 40. In alternate embodiments the beams projected from the additional optic fibers diverge, converge, or are parallel, one to the other. Use of either embodiment shown in FIG. 3 or 4 of the apparatus is essentially the same.

In use of the embodiments of the apparatus of the invention described above, the laser light source connector 26 is first connected to a laser light source. The laser light produced by the laser light source is received by the primary optic fiber proximal end 22 and is transmitted through the length of the primary optic fiber 18, or is received by the plurality of additional optic fibers in the embodiment that does not include a primary optic fiber 18.

The light transmitted through the primary optic fiber 18 is emitted from the distal end surface 28 of the fiber where it is received by the proximal end surfaces 30 of the additional optic fibers 34, thus multiplying the beam. The laser light is then transmitted through each of the additional optic fibers 34 to the distal ends 36 of the additional optic fibers. The laser light is projected from the distal end surfaces 38 of the additional optic fibers 34 in the desired pattern for the multiple laser light beams. The multiple laser light beams are directed to the surgical treatment site. In the preferred embodiment, the number of spots impinging on the surgical site is equal to the number of additional optic fibers.

A further embodiment of the apparatus is comprised of the same handle 12 and tip 16, and a similar primary optic fiber 44 having a light source connector 26 at the fiber proximal end 46. However, there are no additional optic fibers. The primary optic fiber 44 extends from the laser light source connector 26 entirely through the handle 12 and through the tip 16. The distal end 48 of the primary optic fiber is positioned adjacent a tapered distal end 50 of the tip 16, and a small portion of the optic fiber projects outwardly from the tip distal end 50.

A pyramid shape is formed onto the distal end portion of the optic fiber. The pyramid shape is formed by polishing four flat surfaces 52 onto the distal end portion of the optic fiber 44, with each flat surface 52 being oriented at an angle relative to the center axis of the optic fiber 44. In the preferred embodiment, the maximum angle is approximately 20 degrees from the optic fiber center axis. In alternate similar embodiments, other numbers of flat surfaces 52 are formed at the distal end of the optic fiber 44.

Laser light travels through the optic fiber 44 and strikes the four flat polished surfaces 52 in the interior of the optic fiber distal end 48. The laser light striking each polished flat surface 52 is reflected by the surface 52 through the interior of the optic fiber distal end 48 to the opposite polished flat surface 52. The reflected laser light strikes the opposite polished flat surface at an angle of incidence that is too great to be reflected, and the light striking the opposite flat surface is refracted through the surface 52 and exits the distal end 48 of the optic fiber as a laser light beam. In this manner, the light traveling through the optic fiber 44 and striking the four polished surfaces 52 produces four individual laser light beams that are emitted from the pyramid shape at the optic fiber distal end 48. Due to the relative angles of the four flat surfaces 52 with the fiber axis, four individual laser light beams diverge slightly from each other and are directed to four spots at the surgical treatment site, producing four desired treatment spots.

A still further embodiment of the apparatus also employs a single primary optic fiber 62 that extends from a laser light source connector 26 at the proximal end 64 of the fiber to the instrument handle 12 and tubular tip 16 at the distal end 66 of the fiber. The optic fiber 62 extends through the handle bore 14 and through the tubular tip 16 to a distal end 66 of the optic fiber 62 positioned adjacent the tip distal end 38. However, as seen in FIG. 9, the distal end 66 of the optic fiber 62 is positioned in the interior bore of the tubular tip 16 at a spaced position from the tip distal end 38. This creates a void area 68 inside the tubular tip 16 between the distal end 66 of the optic fiber 62 and the tip distal end 38.

A cylindrical bushing 72 having a cylindrical center bore 74 is mounted on the distal end 66 of the optic fiber 62. The bushing 72 is inserted into the interior of the tubular tip 16 and holds the optic fiber distal end 66 in a centered position in the interior of the tip 16. The bushing 72 is also spaced by the void 68 from the distal end 38 of the tubular tip 16.

A glass disk 76 is positioned inside the tubular tip 16 at the tip distal end 38. The glass disk 76 has a proximal end surface 78 that faces toward the distal end surface 66 of the optic fiber 62. The void area 68 in the tip 16 separates the glass disk proximal surface 78 from the optic fiber distal end surface 66.

The opposite distal end of the glass disk 76 is formed with a micro lens array that is substantially flush with the distal end 38 of the tubular tip. The micro lens array includes a plurality of lens surfaces 82 that are formed on the distal end surface of the glass disk 76. Each lens surface 82 is a convex surface. Although only four lens surfaces 82 are shown in the drawing figures, a lesser number or a greater number of lens surfaces could be employed.

The spacing provided by the void area 68 in the interior of the tubular tip 16 between the optic fiber distal end 66 and the glass disk proximal surface 78 is adjusted to allow all of the laser energy emitted from the optic fiber distal end surface 66 to be gathered by the array of lens surfaces 82. The four lens surfaces 82 shown in the drawing figures are arranged in a general square pattern (other patterns of lens surfaces may be employed if a different number of treatment spots is desired). The shapes of the lens surfaces 82 fill each quadrant of the glass disk 76 to eliminate any “dead” areas in the lens array.

The lens surfaces 82 are arranged in a pattern where each lens gathers the laser light striking the lens surface 82 in the interior of the micro lens and focuses the laser light into a separate treatment beam. Each treatment beam is directed from a lens surface 82 of the micro lens at an angle to the center axis of the instrument tip. The separate laser light beams directed from each lens surface 82 at the instrument tip are arranged to strike in a pattern of spots at the surgical site to form the desired treatment pattern.

Thus, each of the embodiments of the multiple target laser probe apparatus of the invention provides an ophthalmic surgery instrument that is capable of splitting a single laser beam received from a single laser light source into a multiple of laser beams. In use, the multiple of laser beams are targeted at a multiple of spots of a surgical site in the eye. In this manner, the apparatus of the invention enables a surgeon to target more than one spot at a time. A surgeon will insert the instrument into the eye, positioning the instrument at the surgical site. The laser source will be activated to transmit laser light through the instrument to create multiple spots at the surgical site. In this manner, the surgical procedure that might otherwise be performed using only one laser spot is performed with multiple spots, essentially simultaneously.

While not specifically described, further embodiments of the instrument include additional functionality, such as the ability to provide illumination through one or more of the fibers. Other embodiments include scissors, forceps, a pick, or other means to manipulate tissue.

Although several embodiments of the apparatus of the invention have been described above, it should be understood that modifications and variations could be made to the apparatus, for example, positioning the plurality of additional optic fiber distal ends adjacent each other with the distal end surfaces of the fibers being positioned at angles to project a diverging pattern of laser beams, without departing from the intended scope of the following claims.

What is claimed is:

1) A multiple target ophthalmic surgery apparatus comprising:
an instrument having a handle that is dimensioned to be held and manipulated by a single hand of a user of the instrument and a rigid tubular tip that extends from the handle to a distal end of the tip that is dimensioned for insertion into an eye in performing ophthalmic surgery procedures;
at least one primary optic fiber having a length that extends from the instrument handle to an end of the at least one primary optic fiber, the length of the at least one primary optic fiber enabling the instrument handle to move freely relative to the end of the at least one primary optic fiber;
a light source connector on the end of the at least one primary optic fiber, the light source connector being connectable to a separate light source to transmit light from the light source through the at least one primary optic fiber to the instrument handle; and,
a plurality of additional optic fibers having lengths that extend through the instrument tip to distal ends of the plurality of additional optic fibers having distal end surfaces adjacent the distal end of the instrument tip, the distal end surfaces of the plurality of additional optic fibers facing in a plurality of different directions to project light transmitted through the plurality of additional optic fibers in a plurality of different directions.

2) The apparatus of claim 1, further comprising:
the distal end surfaces of the plurality of additional optic fibers facing in diverging directions away from the tubular tip to project light transmitted through the plurality of additional optic fibers in diverging directions.

3) The apparatus of claim 1, further comprising:
at least one spacer connected to the distal end of at least one additional optic fiber of the plurality of additional optic fibers and spacing the distal end of the at least one additional optic fiber from the distal end of an adjacent additional optic fiber.

4) The apparatus of claim 3, further comprising:
the at least one spacer being inside the instrument tip.

5) The apparatus of claim 1, further comprising:
a spacer connected to and spacing the distal ends of adjacent additional optic fibers of the plurality of additional optic fibers.

6) The apparatus of claim 1, further comprising:
a spacer connected to and spacing the distal ends of the plurality of additional optic fibers.

7) The apparatus of claim 1, further comprising:
the primary optic fiber being the only optic fiber that extends from the light source connector to the instrument handle.

8) A multiple target ophthalmic surgery apparatus comprising:
an instrument that is dimensioned to be held and manipulated by a single hand of a user;
a primary optic fiber having a length with opposite proximal and distal ends, the length of the primary optic fiber extending from the proximal end of the primary optic fiber and entering the instrument and extending to the distal end of the primary optic fiber;
a light source connector at the proximal end of the primary optic fiber for connecting the proximal end of the primary optic fiber to a light source for transmitting light through the length of the primary optic fiber to the distal end of the primary optic fiber; and
a plurality of additional optic fibers, each additional optic fiber having a length with opposite proximal and distal ends and having a distal end surface at the additional optic fiber distal end, the proximal ends of the plurality of additional optic fibers being connected to the primary optic fiber distal end to receive light transmitted by the primary optic fiber distal end and transmit light through the lengths of the plurality of additional optic fibers to the distal end surfaces of the plurality of additional optic fibers, and the distal end surfaces of the plurality of additional optic fibers being spaced from each other.

9) The apparatus of claim 8, further comprising:
at least one spacer connected to the distal end of at least one additional optic fiber of the plurality of additional optic fibers and spacing the distal end of the at least one additional optic fiber from the distal end of an adjacent additional optic fiber.

10) The apparatus of claim 8, further comprising:
a spacer connected to and spacing the distal ends of adjacent additional optic fibers of the plurality of additional optic fibers.

11) The apparatus of claim 8, further comprising:
a spacer connected to and spacing the distal ends of the plurality of additional optic fibers.

12) The apparatus of claim 8, further comprising:
the distal end surfaces of the plurality of additional optic fibers being directed to project light transmitted through the plurality of additional optic fibers in a plurality of diverging directions.

13) The apparatus of claim 8, further comprising:
the distal end surfaces of the plurality of additional optic fibers being directed to project light transmitted through the plurality of additional optic fibers in one direction.

14) The apparatus of claim 8, further comprising:
the primary optic fiber being the only optic fiber that extends from the light source connector to the instrument.

15) The apparatus of claim 8, further comprising:
the instrument having a handle that is dimensioned to be held and manipulated by a single hand of a user of the instrument and a rigid tubular tip that extends from the handle to a distal end of the tip that is dimensioned for insertion into an eye in performing ophthalmic surgery procedures; and,
the primary optic fiber extends into the instrument handle and the plurality of additional optic fibers extend through the instrument tip.

16) A multiple target ophthalmic surgery apparatus comprising:
an instrument that is dimensioned to be held and manipulated by a single hand of a user;
a primary optic fiber having a length with opposite proximal and distal ends, the length of the primary optic fiber extending from the proximal end of the primary optic fiber and entering the instrument and extending to the distal end of the primary optic fiber;
a light source connector at the proximal end of the primary optic fiber for connecting the proximal end of the primary optic fiber to a light source for transmitting light through the primary optic fiber to the primary optic fiber distal end; and
a plurality of additional optic fibers in the instrument, the plurality of additional optic fibers having lengths that extend side-by-side with opposite proximal ends and distal ends and with the distal ends having distal end surfaces, the proximal ends of the plurality of additional optic fibers being connected to the distal end of the primary optic fiber to receive light transmitted through the primary optic fiber and to transmit the light through the plurality of additional optic fibers to the distal end surfaces of the plurality of additional optic fibers, and the distal end surfaces of the plurality of additional optic fibers facing in a plurality of diverging directions to project the light transmitted through the plurality of additional optic fibers in diverging directions away from the instrument.

17) The apparatus of claim 16, further comprising:
the primary optic fiber being the only optic fiber that extends from a light source connector into the instrument.

18) The apparatus of claim 16, further comprising:
the instrument having a handle that is dimensioned to be held and manipulated by a single hand of a user of the instrument and a rigid tubular tip that extends from the handle to a distal end of the tip that is dimensioned for insertion into an eye in performing ophthalmic surgery procedures, the primary optic fiber extends into the instrument handle and the plurality of additional optic fibers extend through the instrument tip.

19) The apparatus of claim 16, further comprising:
at least one spacer connected to the distal end of at least one additional optic fiber of the plurality of additional optic fibers and spacing the distal end of the at least one additional optic fiber from the distal end of an adjacent additional optic fiber.

20) The apparatus of claim 16, further comprising:
a spacer connected to and spacing the distal ends of adjacent additional optic fibers of the plurality of additional optic fibers.

21) The apparatus of claim 16, further comprising:
a spacer connected to and spacing the distal ends of the plurality of additional optic fibers.

22) The apparatus of claim 16, further comprising:
the instrument having an instrument handle that is dimensioned to be held and manipulated by a single hand of a user of the instrument, and a rigid tubular tip that extends from the handle to a distal end of the tip that is dimensioned for insertion into an eye in performing ophthalmic surgery procedures, the primary optic fiber extends into the handle and the plurality of additional optic fibers extend through the tip to the distal end surfaces of the plurality of additional optic fibers positioned adjacent the distal end of the tip; and,
a spacer inside the tip and engaging the distal ends of the plurality of additional optic fibers and holding the distal ends of the plurality of additional optic fibers in a spaced relationship.

23) A multiple target ophthalmic surgery apparatus comprising:
an instrument having proximal and distal ends and dimensioned to be held and manipulated by a single hand of a user, the instrument comprising:
a handle portion positioned adjacent the proximal end of the instrument, and
a tip portion positioned adjacent the distal end of the instrument and dimensioned to be inserted into an eye;
at least two optic fibers having proximal and distal ends, at least a portion of the at least two optic fibers extending generally through the instrument such that the distal ends of the at least two optic fibers are positioned adjacent to a distal end of the instrument tip portion; and
a means for connecting the at least two optic fibers to a laser light source in a manner such that a portion of a single beam from the laser light source is transmitted into the at least two optic fibers.

24) The ophthalmic surgery apparatus of claim 23, further comprising:
a primary optic fiber having proximal and distal ends;
a connector attached to the proximal end of the primary optic fiber allowing connection of the primary optic fiber to a laser light source; and
a joint between the distal end of the primary optic fiber and the proximal ends of the at least two optic fibers, the joint allowing transmission of laser light between the primary optic fiber and the at least two optic fibers.

25) The ophthalmic surgery apparatus of claim 24 wherein the joint consists of one of a joint constructed with fused glass, an adhesive, or an index-of-refraction-matching gel.

26) The ophthalmic surgery apparatus of claim 24 wherein the joint comprises an index-of-refraction-matching gel and is contained in a first tube.

27) The ophthalmic surgery apparatus of claim 26 further comprising a second tube surrounding at least a portion of the first tube, such second tube having a material composition that absorbs laser light energy emitted from the joint, and wherein the first tube is generally transparent to the laser light energy emitted from the joint.

28) The ophthalmic surgery apparatus of claim 27 wherein the first tube is glass and the second tube is metal.

29) The ophthalmic surgery apparatus of claim 23 wherein the distal ends of the at least two optic fibers are arranged in different directions so that the laser light emitted from the at least two optic fibers is emitted in different directions.

30) The ophthalmic surgery apparatus of claim 29 wherein the laser light emitted from the at least two optic fibers is emitted in diverging directions.

31) The ophthalmic surgery apparatus of claim 23 wherein the at least two optic fibers consist of a number of fibers selected from the group consisting of two, three, four, five, six, seven, and eight.

32) A method of performing ophthalmic surgery comprising:
inserting a portion of an ophthalmic surgery apparatus into the eye;
transmitting a single laser beam into the ophthalmic surgery apparatus;
splitting the laser beam into multiple beams within the apparatus; emitting multiple beams simultaneously from the apparatus onto the surgical site within the eye.

33) The method of claim 32 wherein the transmitting, splitting and emitting is repeated at multiple surgical sites within the eye.

34) The method of claim 32 wherein the multiple beams emitted onto the surgical site are emitted in diverging directions from the ophthalmic surgery apparatus.
